SEQUENCE LISTING (120 sequences)

SEQ ID NO: 1 - DNA, 21 bp, Artificial
  Sense siRNA strand against WSSV VP28 gene
gguuggaucaggcuacuuctt

SEQ ID NO: 2 - DNA, 21 bp, Artificial
  Antisense siRNA strand against WSSV VP28 gene
gaaguagccugauccaacctc

SEQ ID NO: 3 - DNA, 65 bp, Artificial
  Top strand oligonucleotide template for siRNA
gatccggttggatcaggctacttcttcaagagagaagtagcctgatccaacctcttttttggaaa

SEQ ID NO: 4 - DNA, 65 bp, Artificial
  Bottom strand oligonucleotide template for siRNA
agcttttccaaaaaagaggttggatcaggctacttctctcttgaagaagtagcctgatccaaccg

SEQ ID NO: 5 - DNA, 21 bp, Artificial
  Sense siRNA strand against WSSV VP28 gene
ggcuacuucaagaugacugtt

SEQ ID NO: 6 - DNA, 21 bp, Artificial
  Antisense siRNA strand against WSSV VP28 gene
cagucaucuugaaguagcctg

SEQ ID NO: 7 - DNA, 65 bp, Artificial
  Top strand oligonucleotide template for siRNA
gatccggctacttcaagatgactgttcaagagacagtcatcttgaagtagcctgttttttggaaa

SEQ ID NO: 8 - DNA, 65 bp, Artificial
  Bottom strand oligonucleotide template for siRNA
agcttttccaaaaaacaggctacttcaagatgactgtctcttgaacagtcatcttgaagtagccg

SEQ ID NO: 9 - DNA, 21 bp, Artificial
  Sense siRNA strand against WSSV VP28 gene
gguguggaacaacacaucatt

SEQ ID NO: 10 - DNA, 21 bp, Artificial
  Antisense siRNA strand against WSSV VP28 gene
ugauguguuguuccacacctt

SEQ ID NO: 11 - DNA, 63 bp, Artificial
  Top strand oligonucleotide template for siRNA
gatccggtgtggaacaacacatcattcaagagatgatgtgttgttccacaccttttttggaaa

SEQ ID NO: 12 - DNA, 63 bp, Artificial
  Bottom strand oligonucleotide template for siRNA
agcttttccaaaaaaggtgtggaacaacacatcatctcttgaatgatgtgttgttccacaccg

SEQ ID NO: 13 - DNA, 21 bp, Artificial
  Sense siRNA strand against WSSV VP26 gene
gggcaaagguaaugucaautt

SEQ ID NO: 14 - DNA, 21 bp, Artificial
  Antisense siRNA strand against WSSV VP26 gene
auugacauuaccuuugccctt

SEQ ID NO: 15 - DNA, 63 bp, Artificial
  Top strand oligonucleotide template for siRNA
gatccgggcaaaggtaatgtcaatttcaagagaattgacattacctttgcccttttttggaaa

SEQ ID NO: 16 - DNA, 63 bp, Artificial
  Bottom strand oligonucleotide template for siRNA
agcttttccaaaaaagggcaaaggtaatgtcaattctcttgaaattgacattacctttgcccg

SEQ ID NO: 17 - DNA, 21 bp, Artificial
  Sense siRNA strand against WSSV VP26 gene
gguccuacaauacuccucutt

SEQ ID NO: 18 - DNA, 21 bp, Artificial
  Antisense siRNA strand against WSSV VP26 gene
agaggaguauuguaggacctc

SEQ ID NO: 19 - DNA, 65 bp, Artificial
  Top strand oligonucleotide template for siRNA
gatccggtcctacaatactcctctttcaagagaagaggagtattgtaggacctcttttttggaaa

SEQ ID NO: 20 - DNA, 65 bp, Artificial
  Bottom strand oligonucleotide template for siRNA
agcttttccaaaaaagaggtcctacaatactcctcttctcttgaaagaggagtattgtaggaccg

SEQ ID NO: 21 - DNA, 21 bp, Artificial
  Sense siRNA strand against WSSV VP26 gene
ggaaacauuaagggaaauatt

SEQ ID NO: 22 - DNA, 21 bp, Artificial
  Antisense siRNA strand against WSSV VP26 gene
uauuucccuuaauguuucctg

SEQ ID NO: 23 - DNA, 64 bp, Artificial
  Top strand oligonucleotide template for siRNA
gatccgaaacattaagggaaatattcaagagatatttcccttaatgtttcctgttttttggaaa

SEQ ID NO: 24 - DNA, 62 bp, Artificial
  Bottom strand oligonucleotide template for siRNA
agcttttccaaaaaagaaacattaagggaaatatctcttgaatatttcccttaatgtttccg

SEQ ID NO: 25 - DNA, 21 bp, Artificial
  Sense siRNA strand against WSSV ProIn gene
gggaagaauucuacaagaatt

SEQ ID NO: 26 - DNA, 21 bp, Artificial
  Antisense siRNA strand against WSSV ProIn gene
uucuuguagaauucuuccctg

SEQ ID NO: 27 - DNA, 65 bp, Artificial
  Top strand oligonucleotide template for siRNA
gatccgggaagaattctacaagaattcaagagattcttgtagaattcttccctgttttttggaaa

SEQ ID NO: 28 - DNA, 65 bp, Artificial
  Bottom strand oligonucleotide template for siRNA
agcttttccaaaaaacagggaagaattctacaagaatctcttgaattcttgtagaattcttcccg

SEQ ID NO: 29 - DNA, 21 bp, Artificial
  Sense siRNA strand against WSSV ProIn gene
gggacccuuucaugaaacatt

SEQ ID NO: 30 - DNA, 21 bp, Artificial
  Antisense siRNA strand against WSSV ProIn gene
uguuucaugaaaggguccctt

SEQ ID NO: 31 - DNA, 63 bp, Artificial
  Top strand oligonucleotide template for siRNA
gatccgggaccctttcatgaaacattcaagagatgtttcatgaaagggtcccttttttggaaa

SEQ ID NO: 32 - DNA, 63 bp, Artificial
  Bottom strand oligonucleotide template for siRNA
agcttttccaaaaaagggaccctttcatgaaacatctcttgaatgtttcatgaaagggtcccg

SEQ ID NO: 33 - DNA, 21 bp, Artificial
  Sense siRNA strand against WSSV ProIn gene
ggcauacagaugcccuuuatt

SEQ ID NO: 34 - DNA, 21 bp, Artificial
  Antisense siRNA strand against WSSV ProIn gene
uaaagggcaucuguaugcctt

SEQ ID NO: 35 - DNA, 63 bp, Artificial
  Top strand oligonucleotide template for siRNA
gatccggcatacagatgccctttattcaagagataaagggcatctgtatgccttttttggaaa

SEQ ID NO: 36 - DNA, 63 bp, Artificial
  Bottom strand oligonucleotide template for siRNA
agcttttccaaaaaaggcatacagatgccctttatctcttgaataaagggcatctgtatgccg

SEQ ID NO: 37 - DNA, 21 bp, Artificial
  Sense siRNA strand against WSSV Rr092 gene
ggaagauucaucuguucgatt

SEQ ID NO: 38 - DNA, 21 bp, Artificial
  Antisense siRNA strand against WSSV Rr092 gene
ucgaacagaugaaucuucctg

SEQ ID NO: 39 - DNA, 63 bp, Artificial
  Top strand oligonucleotide template for siRNA
gatccgaagattcatctgttcgattcaagagatcgaacagatgaatcttcctgtttttggaaa

SEQ ID NO: 40 - DNA, 65 bp, Artificial
  Bottom strand oligonucleotide template for siRNA
agcttttccaaaaaacaggaagattcatctgttcgatctcttgaatcgaacagatgaatcttccg

SEQ ID NO: 41 - DNA, 21 bp, Artificial
  Sense siRNA strand against WSSV Rr092 gene
ggacaugauuaugcgugugtt

SEQ ID NO: 42 - DNA, 21 bp, Artificial
  Antisense siRNA strand against WSSV Rr092 gene
cacacgcauaaucaugucctg

SEQ ID NO: 43 - DNA, 65 bp, Artificial
  Top strand oligonucleotide template for siRNA
gatccggacatgattatgcgtgtgttcaagagacacacgcataatcatgtcctgttttttggaaa

SEQ ID NO: 44 - DNA, 65 bp, Artificial
  Bottom strand oligonucleotide template for siRNA
agcttttccaaaaaacaggacatgattatgcgtgtgtctcttgaacacacgcataatcatgtccg

SEQ ID NO: 45 - DNA, 21 bp, Artificial
  Sense siRNA strand against WSSV Rr092 gene
ggauaccaucaauagaaagtt

SEQ ID NO: 46 - DNA, 21 bp, Artificial
  Antisense siRNA strand against WSSV Rr092 gene
cuuucuauugaugguaucctt

SEQ ID NO: 47 - DNA, 63 bp, Artificial
  Top strand oligonucleotide template for siRNA
gatccggataccatcaatagaaagttcaagagactttctattgatggtatccttttttggaaa

SEQ ID NO: 48 - DNA, 63 bp, Artificial
  Bottom strand oligonucleotide template for siRNA
agcttttccaaaaaaggataccatcaatagaaagtctcttgaactttctattgatggtatccg

SEQ ID NO: 49 - DNA, 21 bp, Artificial
  Sense siRNA strand against WSSV DNAPol gene
ggaaguggucaucuacgactt

SEQ ID NO: 50 - DNA, 21 bp, Artificial
  Antisense siRNA strand against WSSV DNAPol gene
gucguagaugaccacuucctt

SEQ ID NO: 51 - DNA, 63 bp, Artificial
  Top strand oligonucleotide template for siRNA
gatccggaagtggtcatctacgacttcaagagagtcgtagatgaccacttccttttttggaaa

SEQ ID NO: 52 - DNA, 63 bp, Artificial
  Bottom strand oligonucleotide template for siRNA
agcttttccaaaaaaggaagtggtcatctacgactctcttgaagtcgtagatgaccacttccg

SEQ ID NO: 53 - DNA, 21 bp, Artificial
  Sense siRNA strand against WSSV DNAPol gene
ggaagaacaugaaacuguctt

SEQ ID NO: 54 - DNA, 21 bp, Artificial
  Antisense siRNA strand against WSSV DNAPol gene
gacaguuucauguucuucctt

SEQ ID NO: 55 - DNA, 63 bp, Artificial
  Top strand oligonucleotide template for siRNA
gatccggaagaacatgaaactgtcttcaagagagacagtttcatgttcttccttttttggaaa

SEQ ID NO: 56 - DNA, 63 bp, Artificial
  Bottom strand oligonucleotide template for siRNA
agcttttccaaaaaaggaagaacatgaaactgtctctcttgaagacagtttcatgttcttccg

SEQ ID NO: 57 - DNA, 21 bp, Artificial
  Sense siRNA strand against WSSV DNAPol gene
ggagcauugucauuuaauatt

SEQ ID NO: 58 - DNA, 21 bp, Artificial
  Antisense siRNA strand against WSSV DNAPol gene
uauuaaaugacaaugcucctc

SEQ ID NO: 59 - DNA, 65 bp, Artificial
  Top strand oligonucleotide template for siRNA
gatccggagcattgtcatttaatattcaagagatattaaatgacaatgctcctcttttttggaaa

SEQ ID NO: 60 - DNA, 65 bp, Artificial
  Bottom strand oligonucleotide template for siRNA
agcttttccaaaaaagaggagcattgtcatttaatatctcttgaatattaaatgacaatgctccg

SEQ ID NO: 61 - DNA, 21 bp, Artificial
  Sense siRNA strand against Taura syndrome virus RdRp gene
ggagugucuaaugcggagatt

SEQ ID NO: 62 - DNA, 21 bp, Artificial
  Antisense siRNA strand against Taura syndrome virus RdRp gene
ucuccgcauuagacacucctg

SEQ ID NO: 63 - DNA, 65 bp, Artificial
  Top strand oligonucleotide template for siRNA
gatccggagtgtctaatgcggagattcaagagatctccgcattagacactcctgttttttggaaa

SEQ ID NO: 64 - DNA, 65 bp, Artificial
  Bottom strand oligonucleotide template for siRNA
agcttttccaaaaaacaggagtgtctaatgcggagatctcttgaatctccgcattagacactccg

SEQ ID NO: 65 - DNA, 21 bp, Artificial
  Sense siRNA strand against Taura syndrome virus RdRp gene
gggaagagcggaaagcagatt

SEQ ID NO: 66 - DNA, 21 bp, Artificial
  Antisense siRNA strand against Taura syndrome virus RdRp gene
ucugcuuuccgcucuuccctt

SEQ ID NO: 67 - DNA, 63 bp, Artificial
  Top strand oligonucleotide template for siRNA
gatccgggaagagcggaaagcagattcaagagatctgctttccgctcttcccttttttggaaa

SEQ ID NO: 68 - DNA, 63 bp, Artificial
  Bottom strand oligonucleotide template for siRNA
agcttttccaaaaaagggaagagcggaaagcagatctcttgaatctgctttccgctcttcccg

SEQ ID NO: 69 - DNA, 21 bp, Artificial
  Sense siRNA strand against Taura syndrome virus RdRp gene
ggaauucauuguugacaactt

SEQ ID NO: 70 - DNA, 21 bp, Artificial
  Antisense siRNA strand against Taura syndrome virus RdRp gene
guugucaacaaugaauucctc

SEQ ID NO: 71 - DNA, 65 bp, Artificial
  Top strand oligonucleotide template for siRNA
gatccggaattcattgttgacaacttcaagagagttgtcaacaatgaattcctcttttttggaaa

SEQ ID NO: 72 - DNA, 65 bp, Artificial
  Bottom strand oligonucleotide template for siRNA
agcttttccaaaaaagaggaattcattgttgacaactctcttgaagttgtcaacaatgaattccg

SEQ ID NO: 73 - DNA, 21 bp, Artificial
  Sense siRNA strand against Taura syndrome virus vp1 gene
ggauuggaugagaugucuatt

SEQ ID NO: 74 - DNA, 21 bp, Artificial
  Antisense siRNA strand against Taura syndrome virus vp1 gene
uagacaucucauccaaucctt

SEQ ID NO: 75 - DNA, 63 bp, Artificial
  Top strand oligonucleotide template for siRNA
gatccggattggatgagatgtctattcaagagatagacatctcatccaatccttttttggaaa

SEQ ID NO: 76 - DNA, 63 bp, Artificial
  Bottom strand oligonucleotide template for siRNA
agcttttccaaaaaaggattggatgagatgtctatctcttgaatagacatctcatccaatccg

SEQ ID NO: 77 - DNA, 21 bp, Artificial
  Sense siRNA strand against Taura syndrome virus vp1 gene
gguacgcuugcuaaagcagtt

SEQ ID NO: 78 - DNA, 21 bp, Artificial
  Antisense siRNA strand against Taura syndrome virus vp1 gene
cugcuuuagcaagcguacctg

SEQ ID NO: 79 - DNA, 65 bp, Artificial
  Top strand oligonucleotide template for siRNA
gatccggtacgcttgctaaagcagttcaagagactgctttagcaagcgtacctgttttttggaaa

SEQ ID NO: 80 - DNA, 65 bp, Artificial
  Bottom strand oligonucleotide template for siRNA
agcttttccaaaaaacaggtacgcttgctaaagcagtctcttgaactgctttagcaagcgtaccg

SEQ ID NO: 81 - DNA, 21 bp, Artificial
  Sense siRNA strand against Taura syndrome virus vp1 gene
ggauacgaaggugucuuugtt

SEQ ID NO: 82 - DNA, 21 bp, Artificial
  Antisense siRNA strand against Taura syndrome virus vp1 gene
caaagacaccuucguaucctg

SEQ ID NO: 83 - DNA, 65 bp, Artificial
  Top strand oligonucleotide template for siRNA
gatccggatacgaaggtgtctttgttcaagagacaaagacaccttcgtatcctgttttttggaaa

SEQ ID NO: 84 - DNA, 65 bp, Artificial
  Bottom strand oligonucleotide template for siRNA
agcttttccaaaaaacaggatacgaaggtgtctttgtctcttgaacaaagacaccttcgtatccg

SEQ ID NO: 85 - DNA, 21 bp, Artificial
  Sense siRNA strand against YHV YHVgp gene
ggcucgcauaucauuuauatt

SEQ ID NO: 86 - DNA, 21 bp, Artificial
  Antisense siRNA strand against YHV YHVgp gene
uauaaaugauaugcgagcctg

SEQ ID NO: 87 - DNA, 65 bp, Artificial
  Top strand oligonucleotide template for siRNA
gatccggctcgcatatcatttatattcaagagatataaatgatatgcgagcctgttttttggaaa

SEQ ID NO: 88 - DNA, 65 bp, Artificial
  Bottom strand oligonucleotide template for siRNA
agcttttccaaaaaacaggctcgcatatcatttatatctcttgaatataaatgatatgcgagccg

SEQ ID NO: 89 - DNA, 21 bp, Artificial
  Sense siRNA strand against YHV YHVgp gene
ggauauccucccgccaacatt

SEQ ID NO: 90 - DNA, 21 bp, Artificial
  Antisense siRNA strand against YHV YHVgp gene
uguuggcgggaggauaucctt

SEQ ID NO: 91 - DNA, 63 bp, Artificial
  Top strand oligonucleotide template for siRNA
gatccggatatcctcccgccaacattcaagagatgttggcgggaggatatccttttttggaaa

SEQ ID NO: 92 - DNA, 63 bp, Artificial
  Bottom strand oligonucleotide template for siRNA
agcttttccaaaaaaggatatcctcccgccaacatctcttgaatgttggcgggaggatatccg

SEQ ID NO: 93 - DNA, 21 bp, Artificial
  Sense siRNA strand against YHV YHVgp gene
ggucuuuguuaugaaguagtt

SEQ ID NO: 94 - DNA, 21 bp, Artificial
  Antisense siRNA strand against YHV YHVgp gene
cuacuucauaacaaagacctt

SEQ ID NO: 95 - DNA, 63 bp, Artificial
  Top strand oligonucleotide template for siRNA
gatccggtctttgttatgaagtagttcaagagactacttcataacaaagaccttttttggaaa

SEQ ID NO: 96 - DNA, 63 bp, Artificial
  Bottom strand oligonucleotide template for siRNA
agcttttccaaaaaaggtctttgttatgaagtagtctcttgaactacttcataacaaagaccg

SEQ ID NO: 97 - DNA, 21 bp, Artificial
  Sense siRNA strand against IHHNV orf1 gene
ggacauacugcauacacgutt

SEQ ID NO: 98 - DNA, 21 bp, Artificial
  Antisense siRNA strand against IHHNV orf1 gene
acguguaugcaguaugucctt

SEQ ID NO: 99 - DNA, 63 bp, Artificial
  Top strand oligonucleotide template for siRNA
gatccggacatactgcatacacgtttcaagagaacgtgtatgcagtatgtccttttttggaaa

SEQ ID NO: 100 - DNA, 63 bp, Artificial
  Bottom strand oligonucleotide template for siRNA
agcttttccaaaaaaggacatactgcatacacgttctcttgaaacgtgtatgcagtatgtccg

SEQ ID NO: 101 - DNA, 21 bp, Artificial
  Sense siRNA strand against IHHNV orf1 gene
gguccaaaucaagacccuatt

SEQ ID NO: 102 - DNA, 21 bp, Artificial
  Antisense siRNA strand against IHHNV orf1 gene
uagggucuugauuuggacctg

SEQ ID NO: 103 - DNA, 65 bp, Artificial
  Top strand oligonucleotide template for siRNA
gatccggtccaaatcaagaccctattcaagagatagggtcttgatttggacctgttttttggaaa

SEQ ID NO: 104 - DNA, 65 bp, Artificial
  Bottom strand oligonucleotide template for siRNA
agcttttccaaaaaacaggtccaaatcaagaccctatctcttgaatagggtcttgatttggaccg

SEQ ID NO: 105 - DNA, 21 bp, Artificial
  Sense siRNA strand against IHHNV orf1 gene
ggacaauauaaagacaaactt

SEQ ID NO: 106 - DNA, 21 bp, Artificial
  Antisense siRNA strand against IHHNV orf1 gene
guuugucuuuauauugucctc

SEQ ID NO: 107 - DNA, 65 bp, Artificial
  Top strand oligonucleotide template for siRNA
gatccggacaatataaagacaaacttcaagagagtttgtctttatattgtcctcttttttggaaa

SEQ ID NO: 108 - DNA, 65 bp, Artificial
  Bottom strand oligonucleotide template for siRNA
agcttttccaaaaaagaggacaatataaagacaaactctcttgaagtttgtctttatattgtccg

SEQ ID NO: 109 - DNA, 21 bp, Artificial
  Sense siRNA strand against IHHNV orf2 gene
ggaucaaguggaccagacctt

SEQ ID NO: 110 - DNA, 21 bp, Artificial
  Antisense siRNA strand against IHHNV orf2 gene
ggucugguccacuugaucctt

SEQ ID NO: 111 - DNA, 63 bp, Artificial
  Top strand oligonucleotide template for siRNA
gatccggatcaagtggaccagaccttcaagagaggtctggtccacttgatccttttttggaaa

SEQ ID NO: 112 - DNA, 63 bp, Artificial
  Bottom strand oligonucleotide template for siRNA
agcttttccaaaaaaggatcaagtggaccagacctctcttgaaggtctggtccacttgatccg

SEQ ID NO: 113 - DNA, 21 bp, Artificial
  Sense siRNA strand against IHHNV orf2 gene
ggaggcacaucauuugagatt

SEQ ID NO: 114 - DNA, 21 bp, Artificial
  Antisense siRNA strand against IHHNV orf2 gene
ucucaaaugaugugccucctg

SEQ ID NO: 115 - DNA, 65 bp, Artificial
  Top strand oligonucleotide template for siRNA
gatccggaggcacatcatttgagattcaagagatctcaaatgatgtgcctcctgttttttggaaa

SEQ ID NO: 116 - DNA, 65 bp, Artificial
  Bottom strand oligonucleotide template for siRNA
agcttttccaaaaaacaggaggcacatcatttgagatctcttgaatctcaaatgatgtgcctccg

SEQ ID NO: 117 - DNA, 21 bp, Artificial
  Sense siRNA strand against IHHNV orf2 gene
ggauacuacuggacuacautt

SEQ ID NO: 118 - DNA, 21 bp, Artificial
  Antisense siRNA strand against IHHNV orf2 gene
auguaguccaguaguaucctt

SEQ ID NO: 119 - DNA, 63 bp, Artificial
  Top strand oligonucleotide template for siRNA
gatccggatactactggactacatttcaagagaatgtagtccagtagtatccttttttggaaa

SEQ ID NO: 120 - DNA, 63 bp, Artificial
  Bottom strand oligonucleotide template for siRNA
agcttttccaaaaaaggatactactggactacattctcttgaaatgtagtccagtagtatccg

(19) United States
(12) Patent Application Publication
(10) Pub. No.: US 2008/0194504 A1
(43) Pub. Date: Aug. 14, 2008

(54) Rna-Mediated Interference to Control Disease in Terrestrial and Aquaculture Animals

(75) Inventors: David J. Kyle, Catonsville, MD (US); Arun K. Dhar, Sykesville, MD (US)

Correspondence Address: DUANE MORRIS, LLP;IP DEPARTMENT, 30 SOUTH 17TH STREET, PHILADELPHIA, PA, 19103-4196, US

(73) Assignee: ADVANCED BIONUTRITION CORPORATION, Columbia, MD (US)

(21) Appl. No.: 10/588,414

(22) PCT Filed: Feb. 4, 2005
(86) PCT No.: PCT/US05/03715

§ 371 Date: Nov. 5, 2007

Related U.S. Application Data
(60) Provisional application No. 60/542,391, filed on Feb. 6, 2004.

(51) Int. Cl. A61K 31/7105; A61P 31/04; A61P 31/10; A61P 31/12

(52) U.S. Cl. 514/44

(57) ABSTRACT

The invention is directed to compositions (e.g., feeds, feed supplements, and therapeutic products) and methods for inhibiting an animal pathogen using RNA-interference technology.

### BACKGROUND OF THE INVENTION

The invention relates generally to the field of disease prevention in agricultural and aquacultural populations.

Disease prevention in agriculture and aquaculture is a major focus to maximize both the health of the animals and the commercial viability of raising these animals. Pharmaceuticals and vaccines have a place in this effort, but a less expensive and less invasive method to cure or prevent the symptoms of specific diseases is needed in order to minimize the discomfort of the animals as well as to maximize profits in their growth.

One example is shrimp aquaculture. Over the past few decades, shrimp (Penaeus sp.) farming has evolved from a subsistence level of farming to a major industry providing jobs directly and indirectly to millions of people around the globe. As the shrimp farming evolves, it is poised with many challenges. Among them, viral diseases are of major concern to shrimp farmers. Over the last few years disease like white spot disease, caused by the white spot syndrome virus (WSSV), have caused severe epizootics in the shrimp fanning regions of Asia, Central and South America resulting colossal losses (Krishna et al., 1997, World Aquaculture 12:14-19; Jory et al., 1999, Aquacult. Mag. 25:83-91). WSSV contains a circular double-stranded DNA genome of ˜300 kb in length, and infects all commercially important species of penaeid shrimp and a number of other crustaceans including crabs and crayfish (Flegel, 1997, World J. Micro. Biotech. 13:433-442; van Hulten et al., 2001, Virology 286:7-22; Yang et al., 2001, J. Virol. 75:11811-11820). Since the initial record of the WSSV in East Asia during 1992 to 1993 (Inouye et al., 1994, Fish Pathol. 9:149-158), a number of WSSV encoded genes such as the capsid genes (van Hulten et al., 2000, J. Gen. Virol. 81:2525-2529; van Hulten et al., 2000, Virology 266:227-236; Zhang et al., 2001, Virus Res. 79:137-144; Chen et al., 2002, Virology 293:44-53; Marks et al., 2003, J. Gen. Virol. 84:1517-1523); a ribonucleotide reductase gene (Tsai et al., 2000, Virology 277:92-99); and the thymidine kinases (Tsai et al., 2000b, Virology 277: 100-110) have been studied in detail. In addition, a highly sensitive detection method based on real-time PCR has also been developed for detecting and quantifying the WSSV (Dhar et al., 2001, J. Clin. Microbiol. 39:2835-2845). However, efforts towards developing therapeutics for white spot disease have been very limited. Only recently, it has been reported that addition of beta-1,3-glucan as an additive to shrimp diet at a level of 10 g per kg for 20 days enhances the immunity of shrimp and subsequent survivability against WSSV infection (Chang et al., 2003, Fish Shellfish Immunol. 15:297-310). Using phage display, Yi et al. (2003, J. Gen. Virol. 84:2545-255) have identified a small 10-mer peptide (2E6) that has a high specificity for WSSV, and blocked WSSV-infection in crayfish. Injection of recombinant WSSV capsid proteins (VP 26 and VP 28) was shown to induce resistance in shrimp (P. japonicus; Namikoshia et al., 2004, Aquaculture 229:25-35).

Shrimp, like other crustaceans, do not have adaptive immunity. Instead they rely on the innate immune response. Although many immune genes involved in bacterial and fungal immunity in invertebrates have been well characterized, very few immune genes involved in viral pathogenesis in shrimp or any other invertebrates are known to date. Therefore, there is an urgent need to isolating and characterizing the immune genes in shrimp and to developing therapeutics to combat viral disease in shrimp.

In recent years, a phenomenon called RNA interference (RNAi) has been used to knocking down the expression of a target gene (both cellular and viral genes as target gene; Xia et al., 2002, Nat. Biotechnol. 20:1006-1010; McCown et al., 2003, Virology 313:514-524; Wilson et al., 2003, Proc. Natl. Acad. Sci. USA 100:2783-2788), without causing global changes in gene expression in cells. RNAi is a phenomenon in which a double stranded RNA (dsRNA) suppresses the expression of a target gene by enhancing the specific degradation of the complementary target mRNA (Hannon, 2002, Nature 418:244-251). The mechanism of RNAi involves recognition of the dsRNA by the enzyme RNase III and its cleavage into 21-23 nucleotide short interfering RNA (siRNA). The siRNA is then incorporated into an RNAi targeting complex known as RNA-induced silencing complex (RISC), and cleave the target mRNA that is homologous to siRNA. This results in rapid degradation of the target mRNA and decrease in protein synthesis (Hannon, 2002, Nature 418:244-251). It has been shown recently that RNA mediated interference can be attained through ingestion of dsRNA in Caenorhabditis elegans (Kamath et al., 2002, Genome Biol. 2:0002.0001-0002.0010).

The goal of this invention is to use the RNAi phenomenon to develop therapeutics for the control of viral and bacterial diseases in shrimp, aquaculture, and terrestrial species. Double stranded RNA is fed orally to animals through diets and their efficiency in preventing the disease is measured. Methods are developed for controlling the white spot syndrome disease in shrimp. Five WSSV genes are used as target genes including a WSSV encoded early expressed gene (ribonucleotide reductase), a protease inhibitor, a DNA polymerase gene, a nucleocapsid gene (VP26) and a capsid gene (VP28). A 21-23 nucleotide long WSSV DNA representing these genes are chemically synthesized, and cloned into a feeding vector (L4440). The recombinant plasmid is used to transform Escherichia coli strain HT₁₁₅DE₃ (carrying IPTG-inducible expression of T7 polymerase, and lacks double-strand-specific RNase III; Kamath et al., 2002, Genome Biol. 2:0002.0001-0002.0010). The recombinant clones carrying WSSV specific genes are sequenced to confirm the identity. The whole bacterial cell or broken cells is mixed with diet and fed to shrimp. Shrimp is challenged orally with live infectious WSSV and the mortality is scored. The mRNA expression of the five WSSV target genes is also measured in the treated and control animals using real-time RT-PCR (Dhar et al., 2003, Arch. Virol. 1148:2381-2396; Dhar et al., 2001, J. Clin. Microbiol. 39:2835-2845) to determine the difference in expression in two treatment groups.

Significant disease challenges also exist in other aquacultured species as well as terrestrial animals. In cows, sheep, and goats slow growing organisms such as the mycobacteria cause extensive damage and destruction of herds. Johne's disease (caused by Mycobacterium paratuberculosis johnii) is an example of type of disease. Other slow growing bacteria that could be treated with this approach are the mycoplasmas. Bovine diseases such as Neospora caninum, brucellosis, tuberculosis, bovine leucosis, and diarrhea could be controlled using the instant invention. Protozoan diseases of animals caused by protozoans such as Cryptosporidia and Giardia could be addressed. Viral diseases such as bovine diarrhea and corona virus could also be addressed. A chronic problem in chicken husbandry is the presence of salmonellidae. In addition there are a number of viral diseases that cause extensive damage, such as infectious bronchitis, exotic Newcastle disease, avian influenza (causing the destruction of millions of birds).

Alternative systems exist that could provide cost advantages for any RNAi product produced in these systems versus bacterial systems. Such systems could be algal (e.g., Synechocystis or Chlorella), fungal (e.g., Aspergillus niger, Neurospora crassa), plant (e.g., tobacco, alfalfa, potato, Arabidopsis), and insects (e.g., T. ni and Spodoptera frugiperda). The molecular tools necessary to produce dsRNA constructs in bacterial systems could easily be adapted to these other organisms and, at least for the purposes of this invention, could provide advantages in production costs of the biomass that would be incorporated into a feed. For the purposes of this invention, focus is placed on the plant system. However, expression tools are well developed in the other systems as evidenced by the following references that are included herein by reference for the tools in fungal systems (el-Enshasy et al., 2001, Appl. Biochem. Biotechnol. 90:57-66; Wiebe et al., 2001, Biotechnol. Bioeng. 76:164-174; Liu et al., 2003, Lett. Appl. Microbiol. 36:358-361), algal (Mayfield et al., 1990, Proc. Natl. Acad. Sci. USA 87:2087-2091; U.S. Pat. No. 5,270,175; U.S. Pat. No. 5,977,437; PCT publication WO 00/73455; Toyomizu et al., 2001, J. Appl. Phycol. 13:209-214; US patent publication no. 2002/0164706; U.S. Pat. No. 6,379,968; Shapira et al., 2002, Plant Physiol. 129:7-12; Ton et al., 2002, FASEB J. 16:A542; Mayfield et al., 2003, Proc. Natl. Acad. Sci. USA 100:438-442), yeast (Guthrie et al., 1991, In: Abelson J, Simon M (eds.) Methods Enzyniol. Academic Press, San Diego, Calif.; Mason et al., 1992, Proc. Natl. Acad. Sci. USA 89:11745-11749; Wery et al., 1997, Gene 184:89-97; Martinez et al., 1998, Antonie Van Leeuwenhoek 73:147-153; Cereghino et al., 1999, Curr. Opin. Biotechnol. 10:422-427; Fischer et al., 1999, Biotechnol. Appl. Biochem. 30(Pt 2):117-120; Cereghino et al., 2000, FEMS Microbiol. Rev. 24:45-66; Cregg et al., 2000, Mol. Biotechnol. 16:23-52), and insects (Schmaljohn et al., 1990, J. Virol. 64:3162-3170; Saliki et al., 1992, J. Gen. Virol. 73:369-374; Jarvis et al., 1998, Curr. Opin. Biotechnol. 9:528-533; Altmann et al., 1999, Glycoconj. J. 16:109-123; Cha et al., 1999, Biotechnol. Bioeng. 65:316-324). In the plant system the use of plant viruses as the vehicle for the transfer for genetic material is well developed. Tobacco mosaic virus (TMV) and alfalfa mosaic virus (AMV) are two of these systems with TMV being more wildly utilized. The Ti plasmid based on the T-DNA region from Agrobacterium tumefaciens has been utilized for expression of heterologous DNA in many different plants such as tomato, potato, lupin, and lettuce (Kapusta et al., 1999, FASEB J. 13:1796-1799; Walmsley et al., 2000, Curr. Opin. Biotechnol. 11:126-129; Khandelwal et al., 2003, Plant Sci. 165:77-84).

### BRIEF SUMMARY OF THE INVENTION

It is an object of the invention to produce small interfering RNAs, which are 21-23 nucleotide long, that are specific toward an animal pathogen, such as bacteria, fungi, algae, or yeast. The RNA could be delivered as siRNA or dsRNA that is then processed into the siRNA. The siRNA or progenitor dsRNA would be produced in the cell and either delivered to the animal as the whole cell or as broken cells formulated into the diet. These diets will be fed to the animal to effect oral delivery of therapeutic siRNA specific to the target pathogen to ameliorate the disease or symptoms of the disease.

Longer nucleotides sequence up to around 1000 bases long that are complementary to the target RNA could be produced in the bacterial cell and delivered as whole cell or broken cell mixed with the diet.

It is an object of the invention to protect an animal from pathogen infection by specific degradation of pathogen encoded complementary target mRNA exploiting the RNAi phenomena.

### DETAILED DESCRIPTION OF THE INVENTION

The objects of the invention can be described by the following embodiments.

In one embodiment of the invention a siRNA is produced that is specific to an aquaculture pathogen.

In a further embodiment siRNA produced is specific to a viral aquaculture pathogen.

In a further embodiment siRNA produced is specific to viral pathogens of shrimp.

In a further embodiment siRNA produced is specific for White Spot Syndrome Virus, Yellowhead Virus, Taura Syndrome Virus, and Infectious Hypodermal and Hematopoietic Virus.

In another embodiment the siRNA is specific to viral pathogens of fish.

In a further embodiment the siRNA is specific to Infectious Salmon Anemia Virus, Infectious Pancreatic Necrosis Virus, Carp Spring Viremia Virus, grass carp reovirus, channel catfish virus, channel catfish herpes virus, marine bimavirus, Malbaricus grouper nervous necrosis virus, Dragon grouper nervous necrosis virus, rotaviruses of striped bass, smelt, Atlantic salmon and turbot, viral haemorrhagic septicaemia virus of rainbow trout, rainbow trout rhabdovirus, infectious haematopoietic necrosis virus and sleeping disease virus of rainbow trout.

It is an object of the invention to provide a method of protecting an animal from viral infection comprising the production of a viral pathogen-specific RNA in a bacterial or yeast host, processing the specific RNA containing biomass into a feed or feed supplement with no further purification, providing said feed to the animal to deliver the viral pathogen-specific RNA in an amount sufficient to inhibit the pathogenic effects of virus on the organism.

In one embodiment of the invention a method of protecting an animal from viral infection is provided. The method comprises the steps of: production of a White Spot Syndrome Virus (WSSV)-specific RNA in bacterial host, processing the biomass containing the bacteria that was used to produce the WSSV specific RNA into a feed or feed supplement with no further purification, then providing said feed to the animal to deliver the bacteria containing the WSSV-specific RNA in sufficient quantity to prevent action of the virus on the shrimp. This invention provides a rapid protection to a viral disease like white spot disease. This approach can also be applied to other types of diseases in shrimp caused by other viruses, bacteria, fungi, as well as for diseases in other aquaculture species including fish and mollusk suffering from fungal, bacterial and viral diseases. In aquatic invertebrate species, like shrimp, with “a primitive immune systems” the invention provides a method for treatment of both acute and chronic diseases via delivery of preformed RNAi molecule.

In one embodiment of the invention a method of protecting an animal from bacterial infection is provided. The method comprises the steps of production of a bacterial pathogen-specific RNA in a bacterial host, processing the biomass containing the bacteria that was used to produce the bacterial pathogen-specific RNA into a feed or feed supplement with no further purification, providing the processed biomass to the animal to deliver the bacteria containing the bacterial pathogen-specific RNA in an amount up to 1 percent of the total feed content.

In aquatic vertebrate species, like fish, with well-developed immune systems the invention provides a first response method to retard the onset of an acute infection threat, and eliminate chronic infection.

In further embodiments of the invention other types of expression systems, such as, yeast, algae, and fungi are used for the production of the pathogen-specific RNA. The methods used for the protection of the animal are similar to the embodiments described above.

### DEFINITIONS

In order to fully understand the invention the following definitions are provided to clarify terms of the art that are either variously understood or currently the subject of intense research that might change the scope of the invention if not well defined herein. For this invention the following terms are used as defined herein.

“Gene silencing” a novel gene regulatory mechanism that limits the transcript level by either suppressing transcription (known as transcriptional gene silencing) or by activating a sequence-specific RNA degradation process (posttranscriptional gene silencing).

“RNA interference” or “RNAi” a mechanism of gene silencing that limits the transcript level by a sequence-specific RNA degradation process.

“Small interfering RNA” or “siRNA” are double stranded RNAs that are less than 1000 bases.

“Antisense RNA” single stranded RNA or anticoding strand of RNA serves as a template for synthesis of mRNA.

“Sense RNA” serves as a template for production of the protein or is the same sequence as the mRNA used as the template for protein synthesis.

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

### Construction of recombinant plasmid containing oligonucleotides composed of between 21 and 23 nucleotides homologous to White Spot Syndrome Virus (WSSV) genes.

RNAI sequences were designed by Ambion, Inc. (Austin, Tex.) using their proprietary siRNA design algorithm. RNAi sequences were designed for five genes of white spot syndrome virus (WSSV) including the genes that encode for a nucleocapsid protein VP26, a capsid protein VP 28, a ribonucleotide reductase enzyme, a DNA polymerase enzyme, and a protein that contains Kunitz protease inhibitor signature motif. All five WSSV siRNAs were designed based on the WSSV genomic sequence available in the GenBank database, accession number AF369029. In addition, siRNA was also designed for a non-structural gene and a capsid gene for the infectious hypodermal and haematopoietic necrosis virus (GenBank database, accession number AF273215); a glycoprotein gene of yellowhead virus (GenBank database, accession number AF540644); a RNA-dependent-RNA-polymerase, and a capsid protein gene, VP1 of the Taura syndrome virus (GenBank database, accession number AF277675).

The siRNA design for WSSV gene VP28 (AF369029) can be done several different ways. As designed by Ambion, one siRNA Design is based on a sense siRNA strand (5′→3′) GGUUGGAUCA GGCUACWUCT T (SEQ ID NO:______) and an antisense siRNA strand (5′→3′) GAAGUAGCCU GAUCCAACCT C (SEQ ID NO:______). The template design for this to use pSilencer™ siRNA expression vectors (2.0, 2.1, 3.0, & 3.1 from Ambion) is the top Strand Oligonucleotide Template 5′-GATCCGGTTG GATCAGGCTA CTTCTTCAAG AGAGAAGTAG CCTGATCCAA CCTCTTTTTT GGAAA-3′ (SEQ ID NO:______). With the bottom Strand Oligonucleotide Template: 5′-AGCTTTTCCA AAAAAGAGGT TGGATCAGGC TACTTCTCTC TTGAAGAAGT AGCCTGATCC AACC G-3′ (SEQ ID NO:______).

A second VP28 siRNA design has a sense siRNA strand (5′→3′) GGCUACUUCA AGAUGACUGT T (SEQ ID NO:______) with an antisense siRNA strand (5′→3′) CAGUCAUCUU GAAGUAGCCT G (SEQ ID NO:______). For the pSilencer vectors the top strand oligonucleotide template is 5′-GATCCGGCTA CTTCAAGATG ACTGTTCAAG AGACAGTCA TCTTGAAGTA GCCTGTTTTT TGGAAA-3′ (SEQ ID NO:______) while the bottom strand oligonucleotide template is 5′AGCTTTTCCA AAAAACAGGC TACTTCAAGA TGACTGTCT CTTGAACAGT CATCTTGAAG TAGCC G-3′ (SEQ ID NO:______).

A third possible VP28 siRNA Design has a sense siRNA strand (5′→3′) GGUGUGGAAC AACACAUCAT T (SEQ ID NO:______) and an antisense siRNA strand (5′→3′) UGAUGUGUUG UUCCACACCT T (SEQ ID NO:______). This requires a template design for pSilencer™ siRNA expression vectors having a top strand oligonucleotide template 5′-GATCCGGTGT GGAACAACAC ATCATTCAAG AGA TGATGT GTTGTTCCAC ACCTTTTTTG GAAA-3′ (SEQ ID NO:______) with a bottom strand oligonucleotide template 5′-AGCTTTTCCA AAAAAGGTGT GGAACAACAC ATCATCTCTT GAA TGATGT GTTGTTCCAC ACC G-3′ (SEQ ID NO:______).

The siRNA design for WSSV gene VP26 (AF369029) can be done several different ways. As designed by Ambion one siRNA design is based on a sense siRNA strand (5′→3′) GGGCAAAGGU AAUGUCAAUT T (SEQ ID NO:______) with an antisense siRNA Strand (5′→3′) AUUGACAUUA CCUUUGCCCT T (SEQ ID NO:______). The template design for pSilencer™ siRNA expression vectors (2.0, 2.1, 3.0, & 3.1) has a top strand oligonucleotide template (5′→3′) 5′-GATCCGGGCA AAGGTAATGT CAAT TTCAA GAGAATTGAC ATTACCTTTG CCCTTTTTTG GAAA-3′ (SEQ ID NO:______) with a bottom strand oligonucleotide template (5′→3′) 5′-AGCTTTTCCA AAAAA GGGC AAAGGTAATG TCAATTCTCT TGAAATTGAC ATTACCTTTG CCC G-3′ (SEQ ID NO:______).

A second possible siRNA design for VP26 has a sense siRNA strand (5′→3′) GGUCCUACAA UACUCCUCUT T (SEQ ID NO:______) with an antisense siRNA strand (5′→3′) AGAGGAGUA UUGUAGGACC TC (SEQ ID NO:______). This has a template design for pSilencer™ siRNA expression vectors (2.0, 2.1, 3.0, & 3.1) with a top strand oligonucleotide template 5′-GATCCGGTCC TACAATACTC CTCTTTCAAG AGA AGAGGA GTATTGTAGG ACCTCTTTTT TGGAAA-3′ (SEQ ID NO:______) and a bottom strand oligonucleotide template 5′-AGCTTTTCCA AAAAAGAGGT CCTACAATAC TCCTCTTCTC TTGAAAGAGG AGTATTGTAG GACC G-3′ (SEQ ID NO:______).

A third possible siRNA design for VP26 has a sense siRNA strand (5′→3′) GGAAACAUUA AGGGAAAUAT T (SEQ ID NO:______) with an antisense siRNA Strand (5′→3′) UAUUUCCCUU AAUGUUUCCT G (SEQ ID NO:______). The template design for pSilencer™ siRNA vectors has a top strand oligonucleotide template 5′-GATCCGAAAC ATTAAGGGAA ATATTCAAGA GATATTTCCC TTAATGTTTC CTG TTTTTT GGAAA-3′ (SEQ ID NO:______) with a bottom strand oligonucleotide template 5′-AGCTTTTCCA AAAAA GAAA CATTAAGGGA AATATCTCTT GAATATTTCC CTTAATGTTT CC G-3′ (SEQ ID NO:______).

Another WSSV gene ProIn (AF369029) has a siRNA design with a sense siRNA strand (5′→3′) GGGAAGAAUU CUACAAGAAT T (SEQ ID NO:______) and an antisense siRNA strand (5′→3′) UUCUUGUAGA AUUCUUCCCT G (SEQ ID NO:______). The template design for pSilencer™ siRNA vectors has a top strand oligonucleotide template (5′→3′) 5′-GATCCGGGAA GAATTCTACA AGAATTCAAG AGATTCTTGT AGAATTCTTCC CTGTTTTTTG GAAA-3′ (SEQ ID NO:______) with a bottom strand oligonucleotide template (5′→3′) 5′-AGCTTTTCCA AAAAACAGGG AAGAATTCTA CAAGAATCTC TTGAATTCTT GTAGAATTCT TCCC G-3′ (SEQ ID NO:______).

A second siRNA Design for ProIn has a sense siRNA strand (5′→3′) GGGACCCUUU CAUGAAACAT T (SEQ ID NO:______) and an antisense siRNA strand (5′→3′) UGUUUCAUGA AAGGGUCCCT T (SEQ ID NO:______). The template design for pSilencer™ siRNA vectors has a top strand oligonucleotide template 5′-GATCCGGGAC CCTTTCATGA AACATTCAAG AGATGTTTCA TGAAAGGGTC CC TTTTTTG GAAA-3′ (SEQ ID NO:______) with a bottom strand oligonucleotide template 5′-AGCTTTTCCA AAAAA GGGAC CCTTTCATGA AACATCTCTT GAATGTTTC ATGAAAGGGT CCC G-3′ (SEQ ID NO:______).

A third siRNA Design for ProIn has a sense siRNA strand (5′→3′) GGCAUACAGA UGCCCUUUAT T (SEQ ID NO:______) and an antisense siRNA strand (5′→3′) UAAAGGGCAU CUGUAUGCCT T (SEQ ID NO:______). The template for pSilencer™ siRNA vectors has a top strand oligonucleotide template (5′→3′) 5′-GATCC GGCAT ACAGATGCCC TTTATTCAAG AGATAAAGGG CATCTGTATG CCTTTTTTGG AAA-3′ (SEQ ID NO:______) and bottom strand oligonucleotide template (5′→3′) 5′-AGCTTTTCC AAAAAA GGCA TACAGATGCC CTTTATCTCT TGAATAAAGG GCATCTGTAT GCC G-3′ (SEQ ID NO:______).

Another potential gene for RNA interference is the white spot virus Rr092 gene (AF369029). A possible siRNA design for Rr092 has a sense siRNA strand (5′→3′) GGAAGAUUCA UCUGUUCGAT T (SEQ ID NO:______) and an antisense siRNA strand (5′→3′) UCGAACAGAU GAAUCUUCCT G (SEQ ID NO:______). The template design for pSilencer™ siRNA vectors has a top strand oligonucleotide template (5′→3′) 5′-GATCC GAAGA TTCATCTGTT CGATTCAAGA GATCGAACAG ATGAATCTTC CTG TTTTTGG AAA-3′ (SEQ ID NO:______) and bottom strand oligonucleotide template 5′-AGCTTTTCCA AAAAA CAGG AAGATTCATC TGTTCGATCT CTTGAATCGA ACAGATGAAT CTTCC G-3′ (SEQ ID NO:______).

A second potential siRNA Design for Rr092 has a sense siRNA strand (5′→3′) GGACAUGAUU AUGCGUGUGT T (SEQ ID NO:______) and an antisense siRNA strand (5′→3′) CACACGCAUA AUCAUGUCCT G (SEQ ID NO:______). The template design for pSilencer™ siRNA vectors has a top strand oligonucleotide template 5′-GATCCGGACA TGATTATGCG TGTGTTCAAG AGACACACGC ATAATCATGT CCTGTTTTTT GGAAA-3′ (SEQ ID NO:______) and a bottom strand oligonucleotide template 5′-AGCTTTTCCA AAAAACAGGA CATGATTATG CGTGTGTCTC TTGAACACAC GCATAATCAT GTCC G-3′ (SEQ ID NO:______).

A third potential siRNA design for Rr092 has a sense siRNA strand (5′→3′) GGAUACCAUC AAUAGAAAGT T (SEQ ID NO:______) and an antisense siRNA strand (5′→3′) CUUUCUAUUG AUGGUAUCCT T (SEQ ID NO:______). Template design for pSilencer™ siRNA vectors with a top strand oligonucleotide template 5′-GATCCGGATA CCATCAATAG AAAGTTCAAG AGACTTTCTA TTGATGGTAT CCTTTTTTGG AAA-3′ (SEQ ID NO:______) and a bottom strand oligonucleotide template 5′-AGCTTTTCCA AAAAAGGATA CCATCAATAG AAAG TCTCT TGAACTTTCT ATTGATGGTA TCC G-3′ (SEQ ID NO:______).

Another WSSV gene that could be regulated by RNAi is the DNAPol (AF369029) gene. A potential siRNA design for DNAPol has a sense siRNA strand (5′→3′) GGAAGUGGUC AUCUACGACT T (SEQ ID NO:______) with an antisense siRNA strand (5′→3′) GUCGUAGAUG ACCACUUCCT T (SEQ ID NO:______). Template design for pSilencer™ siRNA vectors has a top strand oligonucleotide template (5′→3′) 5′-GATCCGGAAG TGGTCATCTA CGACTTCAAG AGAGTCGTAG ATGACCACTT CCTTTTTTGG AAA-3′ (SEQ ID NO:______) and a bottom strand oligonucleotide template (5′→3′) 5′-AGCTTTTCCA AAAAAGGAAG TGGTCATCTA CGACTCTCTT GAAGTCGTAG ATGACCACTT CC G-3′ (SEQ ID NO:______).

A second siRNA Design for DNAPol has a sense siRNA Strand (5′→3′) GGAAGAACAU GAAACUGUCT T (SEQ ID NO:______) and an antisense siRNA strand (5′→3′) GACAGUUUCA UGUUCUUCCT T (SEQ ID NO:______). Template design for pSilencer™ siRNA vectors has a top strand oligonucleotide template 5′-GATCCGGAAG AACATGAAAC TGTC TTCAA GAGAGACAGT TTCATGTTCT TCCTTTTTTG GAAA-3′ (SEQ ID NO:______) and a bottom strand oligonucleotide template 5′-AGCTTTTCCA AAAAAGGAAG AACATGAAAC TGTCTCTCTT GAAGACAGTT TCATGTTCTT CC G-3′ (SEQ ID NO:______).

A third design for siRNA for DNAPol has a sense siRNA strand (5′→3′) GGAGCAUUGU CAUUUAAUAT T (SEQ ID NO:______) with an antisense siRNA strand (5′→3′) UAUUAAAUGA CAAUGCUCCT C (SEQ ID NO:______). Template design for pSilencer™ siRNA vectors has a top strand oligonucleotide template 5′-GATCCGGAGC ATTGTCATTT AATA TTCAAG AGATATTAAA TGACAATGCT CCTCTTTTTT GGAAA-3′ (SEQ ID NO:______) and a bottom strand oligonucleotide template 5′-AGCTTTTCCA AAAAA GAGGA GCATTGTCAT TTAATATCTC TTGAATATTA AATGACAATG CTCC G-3′ (SEQ ID NO:______).

Oligonucleotides of 21-23-mer specific for the following five WSSV genes are custom designed and synthesized: a ribonucleotide reductase, a protease inhibitor, a DNA polymerase gene, a nucleocapsid gene (VP26) and a capsid gene (VP28). The synthesis of sense and antisense oligonucleotides representing these genes are based on the published sequence of the WSSV genome, GenBank accession number AF369029 (van Hulten et al., 2001, Virology 286:7-22). Sense and antisense oligonucleotide sequence are annealed to generate double stranded DNA, and are cloned into a plasmid vector such as L440 (Kamath et al., 2002, Genome Biol. 2:0002.0001-0002.0010) or one of the commercially available plasmids (e.g., pSIREN-DNR from Clonetech or pSILENCER from Ambion). The recombinant plasmid is used to transform Escherichia coli strain HT₁₁₅DE₃ (carrying IPTG inducible expression of T7 polymerase and lacking double-strand-specific RNase III; Kamath et al., 2002, Genome Biol. 2:0002.0001-0002.0010). The recombinant clones carrying WSSV specific genes are sequenced to confirm the identity of the clones. Alternative clones can be made based on different strains of the WSSV such as that described by Yang and colleagues (Yang et al., 2001, J. Virol. 75:11811-11820).

### Example 2

### Bacterial induction for the expression of WSSV dsRNA and formulation of feed.

Escherichia coli strain HT₁₁₅DE₃ carrying IPTG inducible WSSV gene are grown in LB medium containing ampicillin, then induced with IPTG for the expression of WSSV-specific RNA. IPTG induction is empirically optimized to obtain maximum induction of dsRNA expression. Bacterial biomass containing cells which express the WSSV gene or genes are mixed with shrimp feed in a microbound format in beads composed of alginate and starch in a polymeric form. Alternative microbound forms are available such as polylactide (Bootland et al., 2002, In: Harrington K (ed) 4th Intl. Symp. Aquatic Animal Health, New Orleans, p 228), carrageen, alginate (Sultana et al., 2000, Int. J. Food Microbiol. 62:47-55; US patent publication no. 2002/0137723; PCT publication WO 03/103692), and chitosan (US patent publication no. 2001/0055807). Attractants are added to make the beads more palatable to the target species (in the case of shrimp, krill meal would be a good attractant).

### Example 3

### Method for Protection of Shrimp from WSSV Infection

Shrimp are fed either a control diet or a diet containing bacterial biomass containing WSSV-specific dsRNA (Example 2). Animals are challenged with WSSV, and their survivability in response to viral infection is measured. The WSSV load in the control and treatment samples is measured by real-time PCR following published protocol (Dhar et al., 2001, J. Clin. Microbiol. 39:2835-2845). The mRNA expression of the five WSSV target genes is measured in the treated and control animals using real-time RT-PCR to determine the difference in expression in two treatment groups following a published method (Dhar et al., 2003, Arch. Virol. 1148:2381-2396).

### Example 4

### Construction Of Recombinant Plasmid Containing Oligonucleotides Of Between 21 and 23 Nucleotides Homologous to Infectious Hematopoetic Necrosis Virus (IPNV) Genes

RNAi sequences were designed by Ambion, Inc. (Austin, Tex.) using their proprietary siRNA design algorithm. siRNA was also designed for a non-structural gene and a capsid gene for the infectious hypodermal and haematopoietic necrosis virus (GenBank database, accession number AF273215). Oligonucleotides of 21-23-mer specific for the following two IPNV genes are custom designed and synthesized: capsid proteins VP2 and VP3. The synthesis of sense and antisense oligonucleotides representing these genes is based on published sequence of the IPNV genome, GenBank accession number AY283780. Sense and antisense oligonucleotide sequence are annealed to generate double stranded DNA, and are cloned into a plasmid vector L4440 (Kamath et al., 2002, Genome Biol. 2:0002.0001-0002.0010). The recombinant plasmid is used to transform Escherichia coli strain HT₁₁₅DE₃ (carrying IPTG inducible expression of T7 polymerase and lacking double-strand-specific RNase III; Kamath et al., 2002, Genome Biol. 2:0002.0001-0002.0010). The recombinant clones carrying IPNV-specific genes are sequenced to confirm the identity of the clones.

### Example 5

### Bacterial induction for the expression of IPNV dsRNA and formulation of feed

Escherichia coli strain HT₁₁₅DE₃ carrying IPTG inducible IPNV gene(s) are grown in LB medium containing ampicillin, and the induced with IPTG for the expression of IPNV-specific RNA. IPTG induction will be empirically optimized to obtain maximum induction of dsRNA expression. Bacterial biomass containing cells expressing IPNV gene(s) is mixed with shrimp feed in a microbound format in beads composed of alginate and starch in a polymeric form. Alternative microbound forms are available such as polylactide (Bootland et al., 2002, In: Harrington K (ed) 4th Intl. Symp. Aquatic Animal Health, New Orleans, p 228), carrageen, alginate, and chitosan. Attractants are added to make the beads more palatable to the target species (in the case of fish, fishmeal is a good attractant).

### Example 6

### Method for Protection of Salmonids from IPNV Infection

Rainbow trout are fed either a control diet or a diet containing bacterial biomass containing IPNV-specific dsRNA (Example 5). Animals are challenged with IPNV, and their survivability in response to viral infection is measured. The IPNV load in the control and treatment samples will be measured by real-time PCR following a protocol similar to that used for WSSV in shrimp (Dhar et al., 2001, J. Clin. Microbiol. 39:2835-2845). The mRNA expression of the two IPNV target genes is measured in the treated and control animals using real-time RT-PCR to determine the difference in expression in two treatment groups following a protocol similar to measuring cellular gene expression in shrimp (Dhar et al., 2003, Arch. Virol. 1148:2381-2396).

### Example 7

### Construction of Recombinant Plasmid Containing Oligonucleotides of Between 21 and 23 Nucleotides Homologous to ISAV Genes

Oligonucleotides of 21-23-mer specific for the following two ISAV genes are custom designed and synthesized: hemagglutinin (HA) and glycoprotein P3. The synthesis of sense and antisense oligonucleotides representing these genes is based on published sequence of the ISAV genome, GenBank accession numbers AF309075 (Krossoy et al., 2001, J. Gen. Virol. 82:1757-1765) and AJ514403 (Snow et al., 2003, Virus Res. 92:99-105). Sense and antisense oligonucleotide sequence are annealed to generate double stranded DNA, and are cloned into a plasmid vector LA440 (Kamath et al., 2002, Genome Biol. 2:0002.0001-0002.0010). The recombinant plasmid is used to transform Escherichia coli strain HT₁₁₅DE₃ (carrying IPTG inducible expression of T7 polymerase and lacking double-strand-specific RNase III; Kamath et al., 2002, Genome Biol. 2:0002.0001-0002.0010). The recombinant clones carrying these ISAV-specific genes are sequenced to confirm the identity of the clones.

### Example 8

### Bacterial Induction for the Expression of ISAV dsRNA and Formulation of Feed

Escherichia coli strain HT₁₁₅DE₃ carrying IPTG inducible ISAV genes (Example 7) are grown in LB medium containing ampicillin, and are induced with IPTG for the expression of ISAV-specific RNA. IPTG induction is empirically optimized to obtain maximum induction of dsRNA expression. Bacterial biomass expressing HA and/or glycoprotein P3 genes is mixed with salmonid feed in a microbound format in beads composed of alginate and starch in a polymeric form. Alternative microbound forms are available, such as polylactide (Bootland et al., 2002, In: Harrington K (ed) 4th Intl. Symp. Aquatic Animal Health, New Orleans, p 228), carrageen, alginate, and chitosan. Attractants are added to make the beads more palatable to the target species (in the case of salmon, AQUASAVOR® is a good attractant).

### Example 9

### Method for Protection of Salmonids from ISAV Infection

Salmonids, such as salmon and rainbow trout, are fed either a control diet or a diet containing bacterial biomass containing ISAV-specific dsRNA (Example 8). Animals are challenged with ISAV, and their survivability in response to viral infection is measured. The ISAV load in the control and treatment samples is measured by real-time PCR following a method similar to that used for WSSV (Dhar et al., 2001, J. Clin. Microbiol. 39:2835-2845). The mRNA expression of the two ISAV target genes (HA and glycoprotein P3) is measured in the treated and control animals using real-time RT-PCR to determine the difference in expression in two treatment groups following a method similar to that used for WSSV (Dhar et al., 2003, Arch. Virol. 1148:2381-2396).

### Example 10

### Construction of Recombinant Plasmid Containing Oligonucleotides Between 21 and 23 Nucleotides Complementary to Carp Spring Virema Genes

Spring viremia of carp, caused by the spring viremia of carp virus (SVCV), is one of the important viral diseases of common carp. The disease is wide spread in European and Asian carp culture (Ahne et al., 2002, Dis. Aquat. Organ. 52:261-272). Carp infected with SVCV show tissue destruction in kidney, spleen, and liver leading to hemorrhage, loss of water-salt balance and impairment of immune response (Ahne et al., 2002, Dis. Aquat. Organ. 52:261-272). The SVCV genome contains a single molecule of linear, negative-sense, single stranded RNA encoding nucleoprotein (N), phosphoprotein (P), matrix protein (M), glycoprotein (G), and RNA dependent RNA polymerase (L) with a short leader and trailer sequence in 3′→5′ direction (Hoffman et al., 2002, Virus Res. 84:89-100).

Oligonucleotides of 21-23-mer specific for these five SVCV genes (N, P, M, G, and L) are custom designed and synthesized based on the published sequence of SVCV genome, GenBank accession number AJ318079 (Hoffman et al., 2002, Virus Res. 84:89-100). Sense and antisense oligonucleotides are annealed to generate double stranded DNA, and cloned into a plasmid vector L4440 (Kamath et al., 2002, Genome Biol. 2:0002.0001-0002.0010). Escherichia coli strain HT115DE3 (carrying IPTG inducible expression of T7 polymerase, and lacks double-strand-specific RNase III) are transformed using the plasmid (Kamath et al., 2002, Genome Biol. 2:0002.0001-0002.0010). The recombinant clones carrying SVCV specific genes are sequenced to confirm the identity of the clones.

### Example 11

### Bacterial Induction for the Expression of Carp Spring Virema dsRNA and Formulation of Feed

Escherichia coli strain HT₁₁₅DE₃ carrying IPTG inducible SVCV genes are grown in LB medium containing ampicillin, and induced with IPTG for the expression of SVCV-specific RNA. IPTG induction is empirically optimized to obtain maximum induction of dsRNA expression. Bacterial cells expressing SVCV gene are mixed with carp feed in a microbound format in beads composed of alginate and starch in a polymeric form and attractants are added to make the beads palatable to carp.

### Example 12

### Fungal Induction for the Expression of Carp Spring Virema dsRNA, and Formulation of Feed

A deletion mutant is made or a strain is used the lacks dsRNase activity. The fungus used can be chosen either from the fungi imperfecti (yeast such as Saccharomyces cerevisiae, Schizosaccharomyces pombe (Raponi et al., 2003, Nucl. Acids Res. 31:4481-4489) or Phaffia rhodozyma) or the filamentous fungi (e.g., Neurospora crassa; Buxton et al., 1990, Biotechnol. Adv. 8:388-389). Strains of P. rhodozyma are already known that have dsRNA present that is associated with a viral infection (Castillo et al., 1994, Curr. Genet. 26:364-368). For the purposes of this example a dsRNase deficient mutant of S. cerevisiae will be constructed using standard molecular techniques to knockout dsRNase identified with the published yeast genome. This will then be transformed with a vector such as pESC-URA (Stratagene). The main function that needs to be present in the transformation vector is the presence of two strong promoters that can co-express RNAs that are complementary to each other. The pESC-URA vector will be modified to contain two pieces of DNA that are specific for spring carp viremia dsRNA using methods similar to those described in Examples 1 & 2. The yeast will be made competent and then transformed as described in the Stratagene users' manual which is a standard molecular biological technique (Ausubel et al., 1997, In: Short Protocols in Molecular Biology, 3 edn. John Wiley & Sons, Inc., New York). Auxotrophic selection will be used and strains that grow on uracil will contain vector. The yeast mutants will be amplified in a galactose medium to drive the two promoters (Gal1 and Gal10) that are provided in the pESC vectors for insertion of the two sequences. The yeast can then be used as a source of dsRNA specific to SVCV. The yeast can be provided as biomass directly added to the feed or microencapsulated and mixed into the feed to prevent infection.

### Example 13

### Method for Protection of Salmonids from Spring Viremia of Carp Virus Infection

Carp is fed either control diet or diet containing bacteria (Example 11) or fungi (Example 12) expressing dsRNA representing N, P, M, G, and L genes of SVCV. Animals are challenged using SVCV contaminated water since waterborne transmission is believed to be the primary route of infection for SVCV (Ahie et al., 2002, Dis. Aquat. Organ. 52:261-272). After SVCV challenge, animals are maintained at 10 to 17° C. temperature because high mortality occurs at that temperature. The survivability in response to SVCV challenge is recorded and the SVCV load in the control and treatment samples is measured by real-time RT-PCR.

### Example 14

### Construction of Recombinant Plasmid Containing 21-23 Nucleotides of Streptococcus iniae Gene.

S. iniae is an important bacterial pathogen of fish infecting salmonid, tilapines, channel catfish, zebrafish among many other fishes worldwide (Zlotkin et al., 1998, Appl. Environ. Microbiol. 64:4065-4067; Shoemaker et al., 2001, Am. J. Vet. Res. 62:174-177; Neely et al., 2002, Infect. Immun. 70:3904-3914). In addition to fish, S. iniae has also been reported to infect human in North America and Asia (Weinstein et al., 1997, N. Engl. J. Med. 337:589-594; Lau et al., 2003, J. Clin. Microbiol. 41:1004-1009). In aquaculture farms, antibiotics are routinely used to control S. iniae infection. Due to growing concerns regarding widespread use of antibiotics in farm raised terrestrial and aquatic animals, there is an urgent need to develop alternative means of controlling the bacterial diseases. Towards this end, the current invention provides a solution in controlling S. iniae as well as other bacterial disease infection in fish.

Oligonucleotides of 21-23-mer specific for S. iniae cytolysin and lactate oxidase genes are custom designed and synthesized based on published S. iniae sequence (Gibello et al., 1999, Appl. Environ. Microbiol. 65:4346-4350; Fuller et al., 2002, Infect. Immun. 70:5730-5739). Cytolysin is a functional homologue of streptolysin produced by group A streptococcus, and cytolysin expression is required for local tissue necrosis (Fuller et al., 2002, Infect. Immun. 70:5730-5739). Lactate oxidase genes, on the other hand, are involved in 1-lactic acid metabolism in S. iniae. Sense and antisense oligonucleotides representing cytolysin and lactate oxidase genes are annealed to generate double stranded DNA, and cloned into a plasmid vector L4440 before transforming Escherichia coli strain HT115DE3 (carrying IPTG inducible expression of T7 polymerase, and lacks double-strand-specific RNase III; Kamath et al., 2002, Genome Biol. 2:0002.0001-0002.0010). The recombinant clones carrying S. iniae genes are sequenced to confirm the identity of the recombinant clones.

### Example 15

### Bacterial Induction for the Expression of Streptococcus iniae dsRNA, and Formulation of Feed

Escherichia coli strain HT115DE3 carrying IPTG inducible S. iniae genes are grown in LB medium containing ampicillin, and induced with IPTG for the expression of S. iniae specific RNA. The expression of dsRNA is empirically optimized by varying the IPTG concentration in the bacterial media. Bacterial cells expressing S. iniae gene are mixed with fish diet in a microbound format in beads composed of alginate and starch in a polymeric form and attractants are added to make the beads palatable to fish.

### Example 16

### Method for Protection of Fish from Infection by Streptococcus iniae

Striped bass are fed either control diet or diet containing bacteria expressing S. iniae dsRNA. Striped bass are challenged with S. iniae. The survivability in response to S. iniae challenge is then recorded in the control and treated fish.

### Example 17

### Construction of Recombinant Plasmid Containing 21-23 Nucleotides of Porcine Parvovirus (PPV), a Pathogen of Pigs

Oligonucleotides of 21-23-mer specific for the VP1 capsid protein gene of PPV are custom designed and synthesized. The synthesis of sense and antisense oligonucleotides representing these genes is based on the published sequence of the porcine parvovirus genome, GenBank accession number NC001718 (Bergeron et al., 1993, Virology 197:86-98). The major antigenic epitopes of PPV are in the VP2 capsid gene, which is chosen for this design (Kamstrup et al., 1998, Virus Res. 53:163-173). Sense and antisense oligonucleotide sequences are annealed to generate double stranded DNA, and are cloned into a plasmid vector L4440 (Kamath et al., 2002, Genome Biol. 2:0002.0001-0002.0010). The recombinant plasmid is used to transform Escherichia coli strain HT₁₁₅DE₃ (carrying IPTG inducible expression of T7 polymerase, and lacks double-strand-specific RNase III; Kamath et al., 2002, Genome Biol. 2:0002.0001-0002.0010). The recombinant clones carrying PPV specific genes are sequenced to confirm the identity of the clones. These siRNAs are then formulated into feeds and are used for the treatment of pigs to prevent PPV infection using methods described in the previous examples for other animals (Examples 2 and 3).

### Example 18

### Construction of Recombinant Plasmid Containing 21-23 Nucleotides of Exotic Newcastle Disease, a Viral Pathogen of Chickens

Oligonucleotides of 21-23-mer specific for the ENDV capsid protein gene are custom designed and synthesized. The synthesis of sense and antisense oligonucleotides representing these genes is based on published sequence of the canine parvovirus genome, GenBank accession number NC002617 (Sellers et al., 2000, Complete sequence for the B1 strain of Newcastle disease virus. In: U.S. Department of Agriculture/Agriculture Research Services). Sense and antisense oligonucleotide sequence are annealed to generate double stranded DNA, and cloned into a plasmid vector such as L4440 (Kamath et al., 2002, Genome Biol. 2:0002.0001-0002.0010) that contains two promoters for production of RNA. The recombinant plasmid is used to transform Escherichia coli strain HT₁₁₅DE₃ (carrying IPTG inducible expression of T7 polymerase, and lacks double-strand-specific RNase III; Kamath et al., 2002, Genome Biol. 2:0002.0001-0002.0010). The recombinant clones carrying CPV specific genes are sequenced to confirm the identity of the clones. These siRNAs are then formulated into feeds and used for the treatment of chickens to prevent ENDV infection using methods described in the previous examples for other animals (see Example 15 for example).

### Example 19

### Construction of Recombinant Plasmid Containing Oligonucleotides of Between 21 and 23 Nucleotides Long Complementary to Foot and Mouth Disease (FMD), a Viral Pathogen of cows.

Oligonucleotides of 21-23-mer specific for the VP1 capsid protein gene are custom designed and synthesized. The synthesis of sense and antisense oligonucleotides representing these genes is based on published sequence of the canine parvovirus genome, GenBank accession number NC 004915 (Saravanan et al., 2003, Foot-and-mouth disease virus Asia 1 (FMDV-Asia1), In: Molecular Virology, Indian Veterinary Research Institute, India). Alternative sequences for various strains could be used in parallel to provide a broader protection than this single strain of FMD. Sense and antisense oligonucleotide sequence are annealed to generate double stranded DNA, and cloned into a plasmid vector L4440 (Kamath et al., 2002, Genome Biol. 2:0002.0001-0002.0010). The recombinant plasmid are used to transform Escherichia coli strain HT₁₁₅DE₃ (carrying IPTG inducible expression of T7 polymerase, and lacks double-strand-specific RNase III; Kamath et al., 2002, Genome Biol. 2:0002.0001-0002.0010). The recombinant clones carrying CPV specific genes are sequenced to confirm the identity of the clones. These siRNAs are then formulated into feeds and used for the treatment of cows to prevent CPV infection using methods described in the previous examples for other animals (see Examples 15).

### Example 20

### Construction of Recombinant Plasmid Containing 21-23 Nucleotides of Feline Leukemia Virus (FLV), a Viral Pathogen of Cats

Oligonucleotides of 21-23-mer specific for an envelope protein gene are custom designed and synthesized. The synthesis of sense and antisense oligonucleotides representing this gene is based on published sequence of the FeLV segment containing envelope protein genes, GenBank accession number AF403716 (Anderson et al., 2001, J. Virol. 75:10563-10572). Sense and antisense oligonucleotide sequence are annealed to generate double stranded DNA, and are cloned into a plasmid vector L4440 (Kamath et al., 2002, Genome Biol. 2:0002.0001-0002.0010). The recombinant plasmid is used to transform Escherichia coli strain HT₁₁₅DE₃ (carrying IPTG inducible expression of T7 polymerase, and lacks double-strand-specific RNase III; Kamath et al., 2002, Genome Biol. 2:0002.0001-0002.0010). The recombinant clones carrying FeLV-specific genes are sequenced to confirm the identity of the clones. These siRNAs are then formulated into feeds and are used for the treatment of cats to prevent FeLV infection, using methods analogous to those described in the previous examples for other animals (Examples 2 and 3).

### Example 21

### Construction of Recombinant Plasmid Containing 21-23 Nucleotides of Canine Parvovirus (CPV), a Viral Pathogen of Dogs

Oligonucleotides of 21-23-mer specific for the VP1 capsid protein gene are custom designed and synthesized. The synthesis of sense and antisense oligonucleotides representing these genes is based on published sequence of the canine parvovirus genome, GenBank accession number NC001539 (Reed et al., 1988, J. Virol. 62:266-276). The N-terminal region of VP1 is chosen for this design since it has been shown to affect nuclear transport of capsids and efficient infection by this virus (Vihinen-Ranta et al., 2002, J. Virol. 76:1884-1891). Sense and antisense oligonucleotide sequence are annealed to generate double stranded DNA, and are cloned into a plasmid vector L4440 (Kamath et al., 2002, Genome Biol. 2:0002.0001-0002.0010). The recombinant plasmid is used to transform Escherichia coli strain HT₁₁₅DE₃ (carrying IPTG inducible expression of T7 polymerase, and lacks double-strand-specific RNase III; Kamath et al., 2002, Genome Biol. 2:0002.0001-0002.0010). The recombinant clones carrying CPV-specific genes are sequenced to confirm the identity of the clones. These siRNAs are then formulated into feeds and are used for the treatment of dogs to prevent CPV infection, using methods analogous to those described in the previous examples for other animals (Examples 2 and 3).

### Example 22

### Construction of recombinant plasmid containing 21-23 nucleotides of White Spot Syndrome Virus (WSSV) genes for expression in plants.

Oligonucleotides of 21-23-mer specific for the following five WSSV genes are custom designed and synthesized, sense and antisense oligonucleotide sequence are annealed to generate double stranded DNA as described in Example 1. For use in plant systems, a different plasmid is used based on the T-DNA region of the Agrobacterium Ti plasmid. A large number of these plasmids are available which are appropriate for their use in a variety of plant species (insert references). The annealed dsDNA is cloned into a plasmid vector having two promoters on opposite strands such as the lac, trp, or PL promoters using standard methods to both construct the plasmid and to produce the construct (Clark, 1997, Plant Molecular Biology. Springer, Berlin). The recombinant plasmid is used to transform alfalfa cells (Taschner et al., 1994, Virology 203:269-276; Larrick et al., 2001, Biomol. Eng. 18:87-94; Kelemen et al., 2002, Transgenic Res. 11:69-72). The recombinant callus culture carrying WSSV-specific genes are sequenced to confirm the identity of the clones. Callus is used to regenerate genetically engineered plants expressing the WSSV-specific RNA. The plant material is dried gently by air-drying then incorporated in feeds and used for protection of shrimp against WSSV infection in methods analogous to those in Examples 2 & 3.

### Example 23

### Alternative Feed Formulation Using Top Coating

Top coating of feeds with biomass is done using standard feed technology wherein the biomass containing the biomass as described in the examples above (e.g., Examples 2, 5, 8, 11, & 12) is top coated on an existing feed and fed to provide the desired regulation of activity.

### Example 24

### Taura Syndrome Virus siRNA Design

RNAI designs for Taura syndrome virus (TSV) RdRp gene (AF277675) that could be regulated by RNAi can be done several different ways. As designed by Ambion one siRNA design is based on a sense siRNA strand (5′→3′) GGAGUGUCUA AUGCGGAGAT T (SEQ ID NO:______) and an antisense siRNA strand (5′→3′) UCUCCGCAUU AGACACUCCT G (SEQ ID NO:______). The template design for this to use pSilencer™ siRNA vectors has the top strand oligonucleotide template 5′-GATCC GGAGT GTCTAATGCG GAGATTCAAG AGATCTCCGC ATTAGACACT CCTGTTTTTT GGAAA-3′ (SEQ ID NO:______) with the bottom strand oligonucleotide template 5′-AGCTTTTCCA AAAAA CAGGA GTGTCTAATG CGGAGATCTC TTGAATCTCC GCATTAGACA CTCC G-3′ (SEQ ID NO:______).

Another RNAi design for Taura syndrome virus (TSV) RdRp gene (AF277675) that could be regulated by RNAi can be done several different ways. As designed by Ambion one siRNA design is based on a sense siRNA strand (5′→3′) GGGAAGAGCG GAAAGCAGAT T (SEQ ID NO:______) and an antisense siRNA strand (5′→3′) UCUGCUUUCC GCUCUUCCCT T (SEQ ID NO:______). The template design for this to use pSilencer™ siRNA vectors has the top strand oligonucleotide template 5′-GATCC GGGAA GAGCGGAAAG CAGATTCAAG AGATCTGCTT TCCGCTCTTC CCTTTTTTGG AAA-3′ (SEQ ID NO:______) with the bottom strand oligonucleotide template 5′-AGCTTTTCCA AAAAA GGGAA GAGCGGAAAG CAGATCTCTT GAATCTGCTT TCCGCTCTTC CC G-3′ (SEQ ID NO:______).

Another RNAi design for Taura syndrome virus (TSV) RdRp gene (AF277675) that could be regulated by RNAi can be done several different ways. As designed by Ambion one siRNA design is based on a sense siRNA strand (5′→3′) GGAAUUCAUU GUUGACAACT T (SEQ ID NO:______) and an antisense siRNA strand (5′→3′) GUUGUCAACA AUGAAUUCCT C (SEQ ID NO:______). The template design for this to use pSilencer™ siRNA vectors has the top strand oligonucleotide template 5′-GATCC GGAAT TCATTGTTGA CAACTTCAAG AGAGTTGTCA ACAATGAATT CCTCTTTTTT GGAAA-3′ (SEQ ID NO:______) with the bottom strand oligonucleotide template as 5′-AGCTTTTCCA AAAAAGAGGA ATTCATTGTT GACAACTCTC TTGAAGTTGT CAACAATGAA TTCC G-3′ (SEQ ID NO:______).

RNAi designs for Taura syndrome virus (TSV) vp1 gene (AF277675) that could be regulated by RNAi can be done several different ways. As designed by Ambion one siRNA design is based on a sense siRNA strand (5′→3′) GGAUUGGAUG AGAUGUCUAT T (SEQ ID NO:______) and an antisense siRNA strand (5′→3′) UAGACAUCUC AUCCAAUCCT T (SEQ ID NO:______). The template design for this to use pSilencer™ siRNA vectors has the top strand oligonucleotide template 5′-GATCC GGATT GGATGAGATG TCTATTCAAG AGATAGACAT CTCATCCAAT CCTTTTTTGG AAA-3′ (SEQ ID NO:______) with the bottom strand oligonucleotide template 5′-AGCTTTTCCA AAAAAGGATT GGATGAGATG TCTATCTCTT GAATAGACAT CTCATCCAAT CC G-3′ (SEQ ID NO:______).

Another RNAi design for Taura syndrome virus (TSV) vp1 gene (AF277675) that could be regulated by RNAi can be done several different ways. As designed by Ambion one siRNA design is based on a sense siRNA strand (5′→3′) GGUACGCUUG CUAAAGCAGT T (SEQ ID NO:______) and an antisense siRNA strand (5′→3′) CUGCUUUAGC AAGCGUACCT G (SEQ ID NO:______). The template design for this to use pSilencer™ siRNA vectors has the top strand oligonucleotide template 5′-GATCC GGTAC GCTTGCTAAA GCAGTTCAAG AGACTGCTTT AGCAAGCGTA CCTGTTTTTT GGAAA-3′ (SEQ ID NO:______) with the bottom strand oligonucleotide template 5′-AGCTTTTCCA AAAAA CAGGT ACGCTTGCTA AAGCAGTCTC TTGAACTGCT TTAGCAAGCG TACC G-3′ (SEQ ID NO:______).

Another RNAi design for Taura syndrome virus (TSV) vp1 gene (AF277675) that could be regulated by RNAi can be done several different ways. As designed by Ambion one siRNA design is based on a sense siRNA strand (5′→3′) GGAUACGAAG GUGUCUUUGT T (SEQ ID NO:______) and an antisense siRNA strand (5′→3′) CAAAGACACC UUCGUAUCCT G (SEQ ID NO:______). The template design for this to use pSilencer™ siRNA vectors has the top strand oligonucleotide template 5′-GATCC GGATA CGAAGGTGTC TTTGTTCAAG AGACAAAGAC ACCTTCGTAT CCTGTTTTTT GGAAA-3′ (SEQ ID NO:______) with the bottom strand oligonucleotide template 5′-AGCTTTTCCA AAAAA CAGGA TACGAAGGTG TCTTTG TCT CTTGAACAAA GACACCTTCG TATCC G-3′ (SEQ ID NO:______).

### Example 25

### sIRNA Design for Yellow Head Virus of Shrimp

RNAi designs for Yellow head virus (YHV) structural glycoprotein gene YHVgp (AF540644) that could be regulated by RNAi can be done several different ways. As designed by Ambion one siRNA design is based on a sense siRNA strand (5′→3′) GGCUCGCAUA UCAUUUAUAT T (SEQ ID NO:______) and an antisense siRNA strand (5′→3′) UAUAAAUGAU AUGCGAGCCT G (SEQ ID NO:______). The template design for this to use pSilencer™ siRNA vectors has the top strand oligonucleotide template 5′-GATCCGGCTC GCATATCATT TATATTCAAG AGATATAAAT GATATGCGAG CCTGTTTTTT GGAAA-3′ (SEQ ID NO:______) with the bottom strand oligonucleotide template 5′-AGCTTTTCCA AAAAACAGGC TCGCATATCA TTTATATCTC TTGAATATAA ATGATATGCG AGCC G-3′ (SEQ ID NO:______).

Another RNAi design for Yellow head virus (YHV) structural glycoprotein gene YHVgp (AF540644) can be done several different ways. As designed by Ambion one siRNA design is based on a sense siRNA strand (5′→3′) GGAUAUCCUC CCGCCAACAT T (SEQ ID NO:______) and an antisense siRNA strand (5′→3′) UGUUGGCGGG AGGAUAUCCT T (SEQ ID NO:______). The template design for this to use pSilencer™ siRNA vectors has the top strand oligonucleotide template 5′-GATCC GGATA TCCTCCCGCC AACATTCAAG AGATGTTGGC GGGAGGATAT CCTTTTTTGG AAA-3′ (SEQ ID NO:______) with the bottom strand oligonucleotide template 5′-AGCTTTTCCA AAAAA GGATA TCCTCCCGCC AACATCTCTT GAATGTTGGC GGGAGGATAT CC G-3′ (SEQ ID NO:______).

Another RNAi design for Yellow head virus (YHV) structural glycoprotein gene YHVgp (AF540644) can be done several different ways. As designed by Ambion one siRNA design is based on a sense siRNA strand (5′→3′) GGUCUUUGUU AUGAAGUAGT T (SEQ ID NO:______) and an antisense siRNA strand (5′→3′) CUACUUCAUA ACAAAGACCT T (SEQ ID NO:______). The template design for this to use pSilencer™ siRNA vectors is the top strand oligonucleotide template 5′-GATCC GGTCT TTGTTATGAA GTAGTTCAAG AGACTACTTC ATAACAAAGA CCTTTTTTGG AAA-3′ (SEQ ID NO:______) with the bottom strand oligonucleotide template 5′-AGCTTTTCCA AAAAAGGTCT TTGTTATGAA GTAGTCTCTT GAACTACTTC ATAACAAAGA CC G-3′ (SEQ ID NO:______).

### Example 26

### Infectious Hypodermal and Hematopoietic Necrosis Virus (IHHNV) siRNA design

The siRNA design for Infectious hypodermal and hematopoietic necrosis virus (IHHNV) gene orf1 (AF273215) can be done several different ways. As designed by Ambion one siRNA Design is based on a sense siRNA strand (5′→3′) GGACAUACUG CAUACACGUT T (SEQ ID NO:______) and an antisense siRNA strand (5′→3′) ACGUGUAUGC AGUAUGUCCT T (SEQ ID NO:______). The template design for this to use pSilencer™ siRNA expression vectors (2.0, 2.1, 3.0, & 3.1 from Ambion) is the top Strand Oligonucleotide Template 5′-GATCCGGACA TACTGCATAC ACGTTTCAAG AGAACGTGTA TGCAGTATGT CCTTTTTTGG AAA-3′ (SEQ ID NO:______) with the bottom strand oligonucleotide template 5′-AGCTTTTCCA AAAAA GGACA TACTGCATAC ACGTTCTCTT GAAACGTGTA TGCAGTATGT CC G-3′ (SEQ ID NO:______).

A second siRNA design for IHHNV gene orf1(AF273215) can be done several different ways. As designed by Ambion one siRNA Design is based on a sense siRNA strand (5′→3′) GGUCCAAAUC AAGACCCUAT T (SEQ ID NO:______) and an antisense siRNA strand (5′→3′) UAGGGUCUUG AUUUGGACCT G (SEQ ID NO:______). The template design for this to use pSilencer™ siRNA vectors is the top strand oligonucleotide template 5′-GATCC GGTCC AAATCAAGAC CCTATTCAAG AGATAGGGTC TTGATTTGGA CCTGTTTTTT GGAAA-3′ (SEQ ID NO:______) with the bottom strand oligonucleotide template 5′-AGCTTTTCCA AAAAACAGGT CCAAATCAAG ACCCTATCTC TTGAATAGGG TCTTGATTTG GACC G-3′ (SEQ ID NO:______).

A third siRNA design for IHHNV gene orf1(AF273215) can be done several different ways. As designed by Ambion one siRNA Design is based on a sense siRNA strand (5′→3′) GGACAAUAUA AAGACAAACT T (SEQ ID NO:______) and an antisense siRNA strand (5′→3′) GUUUGUCUUU AUAUUGUCCT C (SEQ ID NO:______). The template design for this to use pSilencer™ siRNA vectors is the top strand oligonucleotide template 5′-GATCCGGACA ATATAAAGAC AAACTTCAAG AGAGTTTGTC TTTATATTGT CCTCTTTTTT GGAAA-3′ (SEQ ID NO:______) with the bottom strand oligonucleotide template 5′-AGCTTTTCCA AAAAAGAGGA CAATATAAAG ACAAACTCTC TTGAAGTTTG TCTTTATATT GTCC G-3′ (SEQ ID NO:______).

Another gene that could be regulated by RNAi design for IHHNV gene orf2 (AF273215) can be done several different ways. As designed by Ambion one siRNA Design is based on a sense siRNA strand (5′→3′) GGAUCAAGUG GACCAGACCT T (SEQ ID NO:______) and an antisense siRNA strand (5′→3′) GGUCUGGUCC ACUUGAUCCT T (SEQ ID NO:______). The template design for this to use pSilencer™ siRNA vectors is the top strand oligonucleotide template 5′-GATCCGGATC AAGTGGACCA GACCTTCAAG AGAGGTCTGG TCCACTTGAT CCTTTTTTGG AAA-3′ (SEQ ID NO:______) with the bottom strand oligonucleotide template 5′-AGCTTTTCCA AAAAAGGATC AAGTGGACCA GACCTCTCTT GAAGGTCTGG TCCACTTGAT CC G-3′ (SEQ ID NO:______).

Another RNAi design for IHHNV gene orf2 (AF273215) can be done several different ways. As designed by Ambion one siRNA design is based on a sense siRNA strand (5′→3′) GGAGGCACAU CAUUUGAGAT T (SEQ ID NO:______) and an antisense siRNA strand (5′→3′) UCUCAAAUGA UGUGCCUCCT G (SEQ ID NO:______). The template design for this to use pSilencer™ siRNA vectors is the top strand oligonucleotide template 5′-GATCCGGAGG CACATCATTT GAGATTCAAG AGATCTCAAA TGATGTGCCT CCTGTTTTTT GGAAA-3′ (SEQ ID NO:______) with the bottom strand oligonucleotide template 5′-AGCTTTTCCA AAAAACAGGA GGCACATCAT TTGAGATCTC TTGAATCTCA AATGATGTGC CTCC G-3′ (SEQ ID NO:______).

Another RNAi design for IHHNV gene orf2 (AF273215) can be done several different ways. As designed by Ambion one siRNA design is based on a sense siRNA strand (5′→3′) GGAUACUACUGGACUACAUTT (SEQ ID NO:______) and an antisense siRNA strand (5′→3′) AUGUAGUCCA GUAGUAUCCT T (SEQ ID NO:______). The template design for this to use pSilencer™ siRNA vectors is the top strand oligonucleotide template 5′-GATCCGGATA CTACTGGACT ACATTTCAAG AGAATGTAGT CCAGTAGTAT CCTTTTTTGG AAA-3′ (SEQ ID NO:______) with the bottom strand oligonucleotide template 5′-AGCTTTTCCA AAAAAGGATA CTACTGGACT ACATTCTCTT GAAATGTAGT CCAGTAGTAT CC G-3′ (SEQ ID NO:______).

### Example 27

### Cloning of siRNA in Bacterial Plasmid Vector

Sense and antisense oligonucleotide sequence are annealed to generate double stranded DNA, and will be cloned into a pET Directional TOPO™ Expression vector (Invitrogen, Inc., Carlsbad, Calif.) or pCX-TOPO PuruPro™ Caulobacter plasmid expression vector (Invitrogen, Inc.) or L4440 plasmid vector (Kamath et al. 2002). The recombinant plasmid will be used to transform Escherichia coli strain BL21 Star™ (DE3) One Shot Chemically Competent cells (Invitrogen, Inc.) or Caulobacter crescentus cells or Escherichia coli HT₁₁₅DE₃ cells (Kamath et al. 2001), respectively. The recombinant clones will be sequenced to confirm the identity of the clones. Clones that contain virus-specific genes will be grown in LB medium containing ampicillin and IPTG. The concentration of IPTG will be empirically determined to obtain maximum expression of siRNA. Bacterial biomass containing cells which express siRNA will be mixed with shrimp feed in a microbound format in beads composed of alginate and starch in a polymeric form. Attractants such as krill meal will be added to the feed to make it more palatable to shrimp.

### Example 30

### Diseases of Aquatic and Terrestrial Animals

I. Diseases of aquatic animals

A. Protozoa

i. Amyloodinium ocellatum (dinoflagellate)

ii. Brooklynella hostilis (ciliate)

iii. Cryptocaryon irritans (ciliate)

iv. Ichthyopthirius multifilis (Ich—a ciliate)

v. Chilodonella cyprini (a ciliate)

vi. Hexamita (a flagellate)

B. Microsporea

i. Glugea, Spraguea spp.

C. Myxosporia

i. Henneguya spp.

ii. Myxobolus spp.

D. Platyhelminthes

i. Monogenea (trematode)

ii. Digenea (trematodes)

iii. Cestoda (tapeworms)

E. Nematoda

i. Cappalaria (Eimeria spp.)

F. Crustacea

i. Argulus spp. (Brachyuran)

ii. Lepeophtheirus salmonis (Salmon lice—a copepod)

iii. Learnea spp. (Acanthocephalan)

G. Sporozoa

i. Coccidia spp.

H. Fungi

i. Saprolegnia parasitica (an oomycete)

ii. Aphanomyces astaci (an oomycete)

iii. Ichthyophonus hoferi (fungus-like agent)

iv. Fusarium solani (a hyphomycete)

v. Expophiala salmonis (a hyphomycete)

II. Diseases of terrestrial animals

African horse sickness virus (equine)

African swine fever virus (porcine)

Aujeszky's disease virus (porcine herpesvirus 1)

Avian influenza viruses (poultry)

Babesia caballi and B. equi (equine)

Bacillus anthracis (all)

Bluetongue virus (cattle, sheep, goats, deer)

Brucella melitenis (bovine)

Brucella ovis (ovine)

Brucella suis (porcine)

Burkholdaria (Pseudomonas) mallei equine)

Classical swine fever virus (porcine)

Cochliomya hominivorax (ovines, bovines)

Eastern and western equine encephalomyelitis viruses (equine)

Echinococcus multilocularis and E. granulosus (canine)

Equine infectious anaemia virus (equine)

Foot and mouth disease virus (ovine, bovine)

Histoplasma farciminosum (fungus-like many species)

Horse pox virus (equine)

Mycoplasma agalactiae (bovine, ovine)

Mycoplasma mycoides mycoides

Mycoplasma mycoides var Capri (caprine)

Newcastle disease virus (avian)

Peste de petis ruminants virus

Rabies virus and rabies related viruses as follows:

Duvenhage

Lagos bat

Mokola

European bat lyssaviruses I and II.

Rift Valley Fever virus

Rinderpest virus (ovine, bovine, caprine)

Sheep and goat pox virus

Swine vesicular disease

Teschen disease virus

Theileria annulata (ovine, bovine)

Trichinella spiralis (porcine) a protozoan

Trypanosoma equiperdum, T. evansi & T. theileri (Protozoans)

Venezuelan equine encephalomyelitis virus (equine)

Vesicular stomatitis virus (porcine)

Brucellosis

Chronic Wasting Disease

Equine Infectious anemia

Equine viral artertis

Johnes

Psedorabies

Scrapie

Tuberculosis

The foregoing is a non-exhaustive list of disorders and disorder-causing agents that can be treated or inhibited using the methods and compositions described herein.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A feed, feed supplement, or therapeutic composition comprising siRNA or dsRNA, such siRNA or dsRNA being expressed in an organism, and capable of being processed into siRNA that inhibit an animal pathogen.

2. The feed, feed supplement, or therapeutic composition of claim 1, wherein said pathogen is a virus, bacteria, or fungi.

3. The feed, feed supplement, or therapeutic composition of claim 2, wherein said virus is selected from White Spot Syndrome Virus, Taura Syndrome Virus, Yellow Head Virus, and Infectious Hypodermal and Hematopoietic Virus.

4. The feed, feed supplement, or therapeutic composition of claim 2, wherein said virus is selected from porcine parvovirus (PPV), exotic Newcastle disease, foot and mouth disease (FMD), feline leukemia virus (FLV), canine parvovirus.

5. The feed, feed supplement, or therapeutic composition of claim 2, where said bacterium is selected from aquacultural or agricultural bacterial pathogens.

6. The feed, feed supplement, or therapeutic composition of claim 5, wherein said aquacultural bacterial pathogen is selected from Aeromonas spp., Edwardsiella spp., Flavobacterium spp., Flexibacter spp., Mycobacterium sp., Streptococcus spp., Salmonella spp., Vibrio spp., and Yersinia reuckeri.

7. The feed, feed supplement, or therapeutic composition of claim 5, wherein said agricultural bacterial pathogen is selected from Mycobacterium spp., Enterococcus spp., Streptococcus spp., Salmonella spp., and Yersinia pestis.

8. The feed, feed supplement, or therapeutic composition of claim 2, wherein said fungus is selected from aquacultural or agricultural fungal pathogens.

9. The feed, feed supplement, or therapeutic composition of claim 8, wherein said aquacultural fungal pathogen is selected from Oomycetes fungi, Aphanoyces astaci, and Ichthyophonus spp.

10. The feed, feed supplement, or therapeutic composition of claim 8, wherein said agricultural fungal pathogen is selected from Trychophyton spp., Fusarium solani, and Candida spp.

11. The feed, feed supplement, or therapeutic composition of claim 1, wherein said pathogen is a eukaryote.

12. The feed, feed supplement, or therapeutic composition of claim 10, wherein said eukaryote is selected from ciliates, amoeba, protozoa, microsporea, myxosporia, platyhelminthes, nematodes, and sporozoa.

13. The feed, feed supplement, or therapeutic composition of claim 1, wherein said animal is an aquatic animal.

14. The feed, feed supplement, or therapeutic composition of claim 13, wherein said aquatic animal is a crustacean.

15. The feed, feed supplement, or therapeutic composition of claim 14, wherein said crustacean is selected from shrimp, Artemia, lobster, crab, crayfish, prawn.

16. The feed, feed supplement, or therapeutic composition of claim 13, wherein said aquatic animal is a fish.

17. The feed, feed supplement, or therapeutic composition of claim 16, wherein said fish is selected from salmon, trout, halibut, turbot, striped bass, perch, tilapia, catfish, and carp.

18. The feed, feed supplement, or therapeutic composition of claim 1, wherein said animal is a terrestrial animal.

19. The feed, feed supplement, or therapeutic composition of claim 18, wherein said terrestrial animal is selected from horse, dog, cat, cow, sheep, goat, poultry, birds, mink, pig, hamster, mouse, rabbit, rat, gerbil, and guinea pig.

20. The feed, feed supplement, or therapeutic composition of claim 1, wherein said siRNA or dsRNA is expressed in organisms selected from insect, bacteria, fungi, bacteriophage, plants, and yeast.

21. The feed, feed supplement, or therapeutic composition of claim 20, wherein said organisms are included in the feed as whole or mostly whole cells.

22. The feed, feed supplement, or therapeutic composition of claim 20, wherein said organisms are included in the feed as broken or mostly broken cells.

23. The feed, feed supplement, or therapeutic composition of claim, 20, wherein the organism biomass is encapsulated.

24. The feed, feed supplement, or therapeutic composition of claim 23, wherein the encapsulation is accomplished using material selected from digestible polymer, non-digestible polymer, phospholipids, chitosan and alginate.

25. A method of protecting an animal from a pathogen comprising feeding said animal a feed, further comprising siRNA or dsRNA, wherein said siRNA or dsRNA being expressed in an organism, and said siRNA or dsRNA capable of being processed into siRNA and down regulating the expression of animal pathogen by RNA interference.

26. The method of claim 25, wherein said pathogen is a virus, bacteria, or fungi.

27. The method of claim 26, wherein said virus is selected from White Spot Syndrome Virus, Taura Syndrome Virus, Yellow Head Virus, and Infectious Hypodermal and Hematopoietic Virus.

28. The method of claim 26, wherein said virus is selected from porcine parvovirus (PPV), exotic Newcastle disease, foot and mouth disease (FMD), feline leukemia virus (FLV), canine parvovirus.

29. The feed, feed supplement, or therapeutic composition of claim 26, wherein said bacterium is selected from aquacultural or agricultural bacterial pathogens.

30. The feed, feed supplement, or therapeutic composition of claim 29, wherein said aquacultural bacterial pathogen is selected from Aeromonas spp., Edwardsiella spp., Flavobacterium spp., Flexibacter spp., Mycobacterium sp., Streptococcus spp., Salmonella spp., Vibrio spp., and Yersinia reuckeri.

31. The feed, feed supplement, or therapeutic composition of claim 29, wherein said agricultural bacterial pathogen is selected from Mycobacterium spp., Enterococcus spp., Streptococcus spp., Salmonella spp., and Yersinia pestis.

32. The feed, feed supplement, or therapeutic composition of claim 26, wherein said fungus is selected from aquacultural or agricultural fungal pathogens.

33. The feed, feed supplement, or therapeutic composition of claim 32, wherein said aquacultural fungal pathogen is selected from Oomycetes fungi, Aphanomyces astaci, and Ichthyophonus spp.

34. The feed, feed supplement, or therapeutic composition of claim 32, wherein said agricultural fungal pathogen is selected from Trychophyton spp., Fusarium solani, and Candida spp.

35. The method of claim 25, wherein said pathogen is a eukaryote.

36. The method of claim 35, wherein said eukaryote is selected from ciliates, amoeba, protozoa, microsporea, myxosporia, platyhelminthes, nematodes, and sporozoa.

37. The method of claim 25, wherein said animal is an aquatic animal.

38. The method of claim 37, wherein said aquatic animal is a crustacean.

39. The method of claim 38, wherein said crustacean is selected from shrimp, Artemia, lobster, crab, crayfish, prawn

40. The method of claim 37, wherein said aquatic animal is a fish.

41. The method of claim 40, wherein said fish is selected from salmon, trout, halibut, turbot, striped bass, perch, tilapia, catfish, and carp.

42. The feed, feed supplement, or therapeutic composition of claim 25, wherein said animal is a terrestrial animal.

43. The feed, feed supplement, or therapeutic composition of claim 42, wherein said terrestrial animal is selected from horse, dog, cat, cow, sheep, goat, poultry, birds, mink, pig, hamster, mouse, rabbit, rat, gerbil, and guinea pig.

44. The method of claim 25, wherein said siRNA or dsRNA is expressed in organisms selected from insect, bacteria, fungi, bacteriophage, plants, and yeast.

45. The method of claim 44, wherein said organisms are included in the feed as whole or mostly whole cells.

46. The method of claim 44, wherein said organisms are included in the feed as broken or mostly broken cells.

47. The method of claim, 44, wherein the organism biomass is encapsulated.

48. The method of claim 47, wherein the encapsulation is accomplished using material selected from digestible polymer, non-digestible polymer, phospholipids, chitosan and alginate.

49. The method in claim 25, wherein a feed that is supplemented with a recombinant bacterial cells containing 21 to 23 nucleotide long that selectively degrade the homologous mRNA with a disease-causing element to reduce or alleviate a disease state.
